(19) United States
(12) Patent Application Publication
(10) Pub. No.: US 2009/0234389 A1
(43) Pub. Date: Sep. 17, 2009

(54) Interspinous spinal fixation apparatus

(76) Inventors: Fong-Ying Chuang, Kaohsiung County (TW); Kristine M. Khoo, Studio city, CA (US)

Correspondence Address: BACON & THOMAS, PLLC, 625 SLATERS LANE, FOURTH FLOOR, ALEXANDRIA, VA, 22314-1176, US

(21) Appl. No.: 12/382,209

(22) Filed: Mar. 11, 2009

(30) Foreign Application Priority Data
Mar. 11, 2008 (TW) 97108591

(51) Int. Cl. A61B 17/70

(52) U.S. Cl. 606/249

(57) ABSTRACT

An apparatus for interspinous spinal fixation is disclosed, which includes a main body having an end thereof as a distal end, and another end thereof as a proximal end, a cross section of the main body being of an annular or quasi-annular structure. A first joining point and a second joining point are provided at the distal end that are symmetric to each other relative to an opening of the annular or quasi-annular structure, while a third joining point and a fourth joining point are provided at the proximal end that that are symmetric to each other relative to the opening of the annular or quasi-annular structure. A first lateral piece, a second lateral piece, a third lateral piece, and a fourth lateral piece are joined with the first, the second, the third, and the fourth joining points of the main body, such that the first and the second lateral pieces can rotate axially about the first and the second joining points, thereby rotating from a folded position on the main body to an extended position; and the third and the fourth lateral pieces can rotate axially about the third and the fourth joining points, thereby rotating from a folded position on the main body to an extended position.

### FIELD OF THE INVENTION

The invention relates to an apparatus for stabilizing the human spine, and more particularly to a spinal fixation apparatus to be inserted between neighboring vertebrae.

### DESCRIPTION OF PRIOR ART

Spinal diseases, disorders of the facet joint and related tissues surrounding the spine are often accompanied by severe pain, and such disorders are frequently caused by several factors, which include the degeneration and aging of spinal discs. Moreover, the disorders may further evolve into more serious symptoms like dislocation or herniation of vertebrae. Consequently, any minor movements between the neighboring vertebrae can cause severe pain to an afflicted person while moving around.

Spinal fixation apparatuses to be inserted between vertebrae has been introduced before, including patents like TW-590756, TW-00520986, and TW-00542712; in which a tether was used to securely hold a spinal fixation apparatus between the spinous process and the interspinous space in place. However, an open surgery is required for inserting this type of spinal fixation apparatus, which is more invasive and structurally complicated. Subsequently, the surgery is more time-consuming and leaves a relatively larger wound on the patient.

### SUMMARY OF THE INVENTION

A primary objective of the present invention is to provide an interspinous spinal fixation apparatus.

The interspinous spinal fixation apparatus of the invention stabilizes two neighboring vertebrae by extending lateral pieces thereof.

Another objective of the present invention is to provide a flexible interspinous spinal fixation apparatus.

Another objective of the present invention is to provide an interspinous spinal fixation apparatus that has easily extendable lateral pieces.

Yet another objective of the present invention is to provide an interspinous spinal fixation apparatus having lateral pieces that may be extended by rotating.

Yet another objective of the present invention is to provide an interspinous spinal fixation apparatus having dual-axis lateral pieces that may be extended by rotating.

A further objective of the present invention is to provide an interspinous spinal fixation apparatus that requires less surgery time.

An interspinous spinal fixation apparatus, comprising:

a main body having an end thereof as a distal end, and another end thereof as a proximal end, wherein a cross section of the main body is of an annular or quasi-annular structure, and a first joining point and a second joining point are provided at the distal end that are symmetric to each other relative to an opening of the annular or quasi-annular structure, while a third joining point and a fourth joining point are provided at the proximal end that that are symmetric to each other relative to the opening of the annular or quasi-annular structure; and

at least four lateral pieces, including a first lateral piece, a second lateral piece, a third lateral piece, and a fourth lateral piece, and an end of each of the lateral pieces is an extending end, whereas another end thereof is an joining end, wherein the joining ends of the first, the second, the third, and the fourth lateral pieces are used for joining with the first, the second, the third, and the fourth joining points of the main body, such that the first and the second lateral pieces can rotate axially about the first and the second joining points, thereby rotating from a folded position on the main body to an extended position.

The aforesaid main body may be fitted between the spinous processes of two neighboring vertebrae by modifying sizes and assemblies thereof. The main body may be fabricated by using any suitable materials, and is preferably made of biocompatible materials, such as metals (for example, stainless steel, titanium, aluminum, or metal alloys made of two or more metals), plastics, polymers, rubber, ceramics, natural tissues (such as bones), or composite materials (which are made of two or more than two materials). For judging whether a material is suitable for making the main body or not, a variety of factors may be considered, which include but not limited to the ability of withstanding sterilization, the ability of withstanding forces exerted thereupon, weight, durability, and the ability of combining with the lateral pieces and implantable sleeves.

The aforesaid annular or quasi-annular structure may be an open ring, a closed ring, or a partially open ring, wherein the open ring may be open from an opening of the distal end to an opening of the proximal end of the main body, as indicated in FIG. 7a; whereas the closed ring may be closed from an opening of the distal end to an opening of the proximal end of the main body, which appears as a closed-off O-shaped ring; the partially open ring may be partially closed and partially open from an opening of the distal end to an opening of the proximal end of the main body; the open ring is preferably used among the aforesaid rings.

The aforesaid main body may be any type of cylindrical bodies having a cross section that is an annular or quasi-annular structure. Moreover, a center of the annular or quasi-annular structure is a pivot hole, and the pivot hole may be of any shapes; such as circular, elliptical, or polygonal, and is preferably circular.

The above-mentioned open ring or partially open ring may be any type of cylindrical bodies having a cross section that is a C-shaped ring or quasi C-shaped ring structure. Furthermore, a center of the C-shaped ring or quasi C-shaped ring structure is a pivot hole, and the pivot hole may be of any shapes; such as circular, elliptical, or polygonal, and is preferably circular.

The above-mentioned open ring or partially open ring of the main body also has a gap as an opening, which is used to allow a spot where the spinal fixation apparatus is inserted to be flexibly compressed after surgery.

With regard to the aforesaid main body, the annular or quasi-annular structure at both sides of the distal end thereof may also include a group of correspondingly disposed safeguard mechanisms, which are the first safeguard mechanism and the second safeguard mechanism, wherein the first and the second safeguard mechanisms may be integrally formed with the main body, or integrally combined with the main body by using any of the previously known methods of combination (such as by clasping or screwing), and it is more preferable to have the first and the second safeguard mechanisms integrally formed with the main body, so as to secure the first and the second lateral pieces when they are extended to designated positions.

In regard to the aforesaid main body, the annular or quasi-annular structure at both sides of the proximal end thereof may also include a group of correspondingly disposed safeguard mechanisms, which are the third safeguard mechanism and the fourth safeguard mechanism, wherein the third and the fourth safeguard mechanisms may be integrally formed with the main body, or integrally combined with the main body by using any of the previously known methods of combination (such as by clasping or screwing), and it is more preferable to have the third and the fourth safeguard mechanisms integrally formed with the main body, so as to secure the third and the fourth lateral pieces when they are extended to designated positions.

The aforesaid first, second, third, and fourth safeguard mechanisms may be any of the previously known safeguard methods, such as the use of pins, protruding blocks, and stoppers.

The aforesaid main body may further include a fixing mechanism for securing the main body while rotating the lateral pieces, so that the main body would not become loosened off the interspinous space while the lateral pieces are rotated. In addition, the fixing mechanism may be any of the previously known fixing mechanisms, such as fixing holes for holding clamps that can secure the main body (please see FIG. 1).

The aforesaid main body may further be fitted with an implantable sleeve at the distal end thereof, so as to allow the main body to be inserted into a patient during surgery and firmly secure the lateral pieces. The implantable sleeve may be fabricated by using any suitable materials, and is preferably made of biocompatible materials, such as metals (for example, stainless steel, titanium, aluminum, or metal alloys made of two or more metals), plastics, polymers, rubber, ceramics, or composite materials (which are made of two or more than two materials). For judging whether a material is suitable for making the implantable sleeve or not, a variety of factors may be considered, which include but not limited to the ability of withstanding sterilization, the ability of withstanding forces exerted thereupon, weight, durability, and the ability of combining with the lateral pieces and the main body. Moreover, the implantable sleeve may be integrally formed with the main body, or integrally combined with the main body by using any of the previously known methods of combination (such as by clasping or screwing), and it is more preferable to have the implantable sleeve integrally formed with the main body. The implantable sleeve may be of any shapes, and is preferably of a conical shape.

The aforesaid main body may further be fitted with an implantable sleeve at the proximal end thereof, so as to allow the lateral pieces to be firmly secured after they are rotated to designated positions. The implantable sleeve may be fabricated by using any suitable materials, and is preferably made of biocompatible materials, such as metals (for instance, stainless steel, titanium, aluminum, or metal alloys made of two or more metals), plastics, polymers, rubber, ceramics, or composite materials (which are made of two or more than two materials). For judging whether a material is suitable for making the implantable sleeve or not, a variety of factors may be considered, which include but not limited to the ability of withstanding sterilization, the ability of withstanding forces exerted thereupon, weight, durability, and the ability of combining with the lateral pieces and the main body. Moreover, the implantable sleeve may be integrally combined with the main body by using any of the previously known methods of combination (such as by clasping or screwing).

The aforesaid main body may include four or more than four lateral pieces, wherein the lateral pieces may have the third and the fourth lateral pieces being fixed at the extended position, and have the first and the second lateral pieces being able to be rotated to the extended position; or the first, the second, the third, and the fourth lateral pieces may all be able to be rotated from the folded position to the extended position, such that the lateral pieces may be allowed to surround and hold securely around at least one spinous process from two neighboring vertebrae, and is more preferable to have the first, the second, the third, and the fourth lateral pieces be able to be rotated from the folded position to the extended position.

The aforesaid first and second joining points are correspondingly disposed at the opening of the annular or quasi-annular structure on the distal end of the main body. In addition, the first and the second joining points may be integrally combined with the joining ends of the first and the second lateral pieces by using any of the previously known methods of combination (such as by clasping, screwing, or locking), such that the first and the second lateral pieces may centrally rotate about the first and the second joining points.

The aforesaid third and fourth joining points are correspondingly disposed at the opening of the annular or quasi-annular structure on the proximal end of the main body. Moreover, the third and the fourth joining points may be integrally combined with the joining ends of the third and the fourth lateral pieces by using any of the previously known methods of combination (such as by clasping, screwing, or locking), such that the third and the fourth lateral pieces may be fixed at the extended position or centrally rotated about the third and the fourth joining points, and is more preferable to have the third and the fourth lateral pieces being able to centrally rotate about the third and the fourth joining points.

The first and the third joining points may include a cylindrical hole that passes through the first and the third joining points and penetrates the main body, and a joining rod fitted through the cylindrical hole, wherein the joining ends of the first and the third lateral pieces are connected to two ends of the joining rod by clasping, screwing, and locking, thereby allowing the extending ends of the first and the third lateral pieces to be rotated axially about the joining rod.

The aforesaid second and fourth joining points may include a cylindrical hole that passes through the second and the fourth joining points and penetrates the main body, and a joining rod fitted through the cylindrical hole, wherein the joining ends of the second and the fourth lateral pieces are connected to two ends of the joining rod by clasping, screwing, and locking, thereby allowing the extending ends of the second and the fourth lateral pieces to be rotated axially about the joining rod.

The aforesaid lateral pieces are not limited in shapes thereof, and are preferably in a form of apertures in the annular or quasi-annular structure that does not interfere with the main body. The lateral pieces may be fabricated by using any suitable materials, and is preferably made of biocompatible materials, such as metals (for example, stainless steel, titanium, aluminum, or metal alloys made of two or more metals), plastics, polymers, rubber, ceramics, natural tissues (such as bones), or composite materials (which are made of two or more than two materials). For judging whether a material is suitable for making the lateral pieces or not, a variety of factors may be considered, which include the ability of being bent flexibly and plastically, and/or the ability of allowing the lateral pieces to be deformed, and the ability of retaining any shapes after being bent.

The aforesaid lateral pieces may be further fitted with auxiliary lateral pieces at extending ends thereof, respectively, such that when the lateral pieces are extended, the auxiliary lateral pieces may be further extended, so as to lengthen the lateral pieces, thereby further securing the lateral pieces between neighboring vertebrae.

The aforesaid first, second, third, and fourth lateral pieces may be rotated and extended outwardly from the main body by using an actuator, and then held correspondingly to at least one spinous process of the vertebrae. In addition, the lateral pieces may also include contact surfaces for engaging with opposite facets of two spinous processes of neighboring vertebrae.

The aforesaid actuator may be formed as a key or key-like structure, which is inserted from the proximal end and rotated left and right, so as to allow the extending ends of the lateral pieces to be rotated to the extended position, thereby allowing the lateral pieces to surround and hold securely around at least one spinous process from two neighboring vertebrae.

The invention has proposed an interspinous spinal fixation apparatus having lateral pieces that may be easily rotated, which only requires minimally invasive surgeries for insertion. In addition to being less invasive, it requires shorter surgery duration, and may be flexibly compressed. Therefore, the apparatus may allow for control over vertebrae without critically altering the anatomical structure of the spine, thus preventing patients afflicted with minor symptoms from suffering permanent damages.

### BRIEF DESCRIPTION OF DRAWINGS

The aforesaid objectives and advantages of the present invention will be more clearly understood when considered in conjunction with the accompanying drawings, in which:

FIG. 1 is a perspective view that shows a disassembled interspinous spinal fixation apparatus according to a preferred embodiment of the invention.

FIG. 2a is a perspective view that shows the lateral pieces of FIG. 1 in a folded state.

FIG. 2b is a perspective view that shows the lateral pieces of FIG. 1 in an extended state.

FIG. 3a is a perspective view that shows an interspinous spinal fixation apparatus in a folded state according to another preferred embodiment of the invention.

FIG. 3b is a perspective view that shows an interspinous spinal fixation apparatus in an extended state according to another preferred embodiment of the invention.

FIGS. 4a, 4b, and 4c are close schematic views that show three different shapes of pivot holes of the interspinous spinal fixation apparatus according to another preferred embodiment of the invention.

FIGS. 5a, 5b, and 5c are schematic views that show three different ways for combining the main body and the implantable sleeve according to a further preferred embodiment of the invention.

FIGS. 6a, 6b, and 6c are schematic views that show three different ways for combining the lateral pieces and the joining rod according to a further preferred embodiment of the invention.

FIGS. 7a and 7b are schematic views that show two different ways for combining the lateral pieces and the main body according to a further preferred embodiment of the invention.

FIGS. 8a, 8b, and 8c are schematic views that illustrate three different actuators according to a further preferred embodiment of the invention.

FIG. 9 is a schematic view that illustrates a way for implementing the interspinous spinal fixation apparatus and the actuator according to a further preferred embodiment of the invention.

FIGS. 10a, 10b, 10c, and 10d are schematic views that illustrate the interspinous spinal fixation apparatus being implemented between neighboring vertebrae according to a further preferred embodiment of the invention.

### DETAILED DESCRIPTION OF PREFERRED EMBODIMENTS

The preferred embodiments of the invention will be further elucidated in the following text accompanied with the aforesaid drawings.

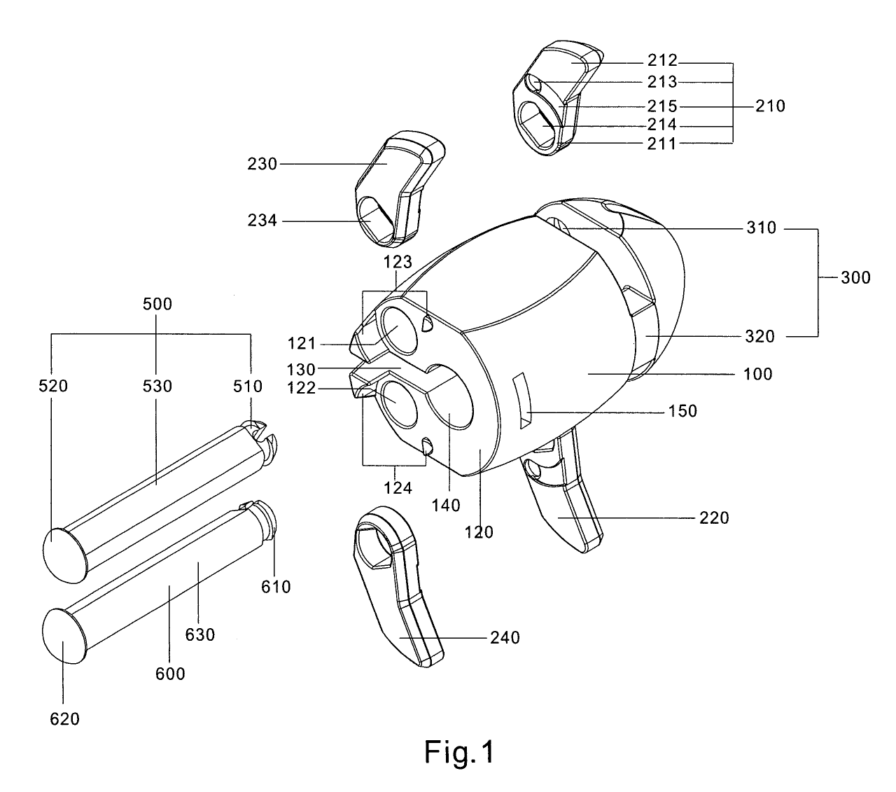

FIG. 1 is a perspective view that shows a disassembled interspinous spinal fixation apparatus according to a preferred embodiment of the invention, which comprises a main body 100. The main body 100 has a proximal end 120; a distal end opposite to the proximal end 120, which cannot be seen in FIG. 1 due to view angle; a third joining point 12 land a fourth joining point 122 of the proximal end 120; a first joining point and a second joining point of the distal end opposite to the third joining point 121 and a fourth joining point 122, which cannot be seen in FIG. 1 due to view angle; a third safeguard mechanism 123 and a fourth safeguard mechanism 124of the proximal end 120; a first safeguard mechanism and a second safeguard mechanism of the distal end opposite to the third safeguard mechanism 123 and the fourth safeguard mechanism 124, which cannot be seen in FIG. 1 due to view angle; an opening 130; a pivot hole 140; a fixing mechanism 150, which is a fixing hole for securely holding clamps; a first lateral piece 210; a second lateral piece 220; a third lateral piece 230; and a fourth lateral piece 240. The first lateral piece 210 has a joining end 211, an extending end 212, a securing hole 213, a joining hole 214, and a groove 215. The third later piece has a joining hole 234. The joining hole 214 has parallel upper and lower surfaces, and left and right surfaces that are arc-shaped for combining with a joining rod. The second lateral piece 220, the third lateral piece 230, and the fourth lateral piece 240 are structurally identical to the first lateral piece 210. The interspinous spinal fixation apparatus shown in FIG. 1 further comprises an implantable sleeve 300; a first joining rod 500; and a second joining rod 600. The sleeve 300 has a first connecting hole 310, a second connecting hole (which cannot be seen in FIG. 1 due to the blocking of the main body 100), and a connecting end 320, wherein the implantable sleeve 300 is connected to the main body 100 via the connecting end 320. The first joining rod 500 has an upper joining end 510; a lower joining end 520; and a rod body 530 therebetween, wherein the rod body 530 has two opposite sides formed as planar surfaces, so as to allow the rod body 530 to be fittingly combined with the first lateral piece 210 and the third lateral piece 230, and prevent the first lateral piece 210 and the third lateral piece 230 from loosening during rotation. The second joining rod 600 is structurally identical to the first joining rod 500, which has an upper joining end 610; a lower joining end 620; and a rod body 630 therebetween. Moreover, the first joining rod 500 may pass through the joining hole 234 of the third lateral piece 230, continuously through the third joining point 121 and the first joining point of the main body 100, and through the joining hole 214 of the first lateral piece 210, thereby allowing a fastener at the upper joining end 510 of the first joining rod 500 to be combined with the first connecting hole 310 of the implantable sleeve 300. The combination between the second lateral piece 220, the fourth lateral piece 240, the main body 100, the second joining rod 600 and the implantable sleeve 300 are identical to that between the first lateral piece 210, the third lateral piece 230, the main body 100, the first joining rod 500 and the implantable sleeve 300. When the third lateral piece 230 is rotated and extended to an extended position, the third safeguard mechanism 123 is used to secure the third lateral piece 230 at the extended position. Furthermore, safeguard mechanisms of the first lateral piece 210 and the second lateral piece 220 (now shown in the drawings), and the safeguard mechanism 124 of the fourth lateral piece 240 are structurally identical to the third safeguard mechanism 123. Similarly, each of the first lateral piece 210, the fourth later piece 240, and the second lateral piece 220 can be rotated and extended to an extended position, and can be secured at the extended position by the associated safeguard mechanism.

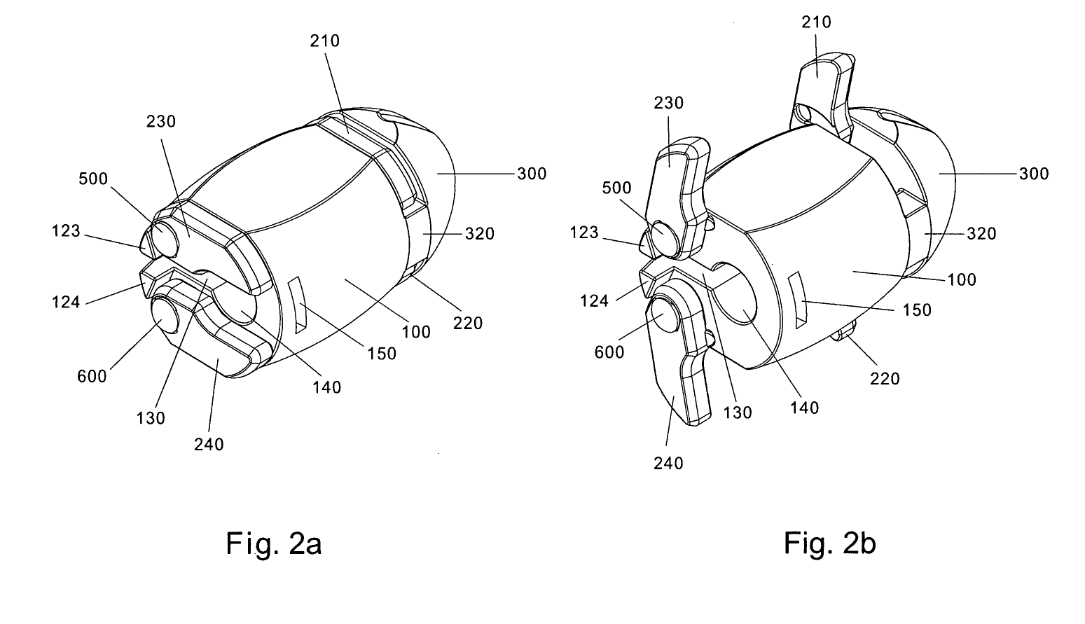

FIG. 2a is a perspective view that shows the lateral pieces of FIG. 1 in a folded state, in which the number 100 indicates the main body; 130 indicates the opening; 140 indicates the pivot hole; 150 indicates the fixing mechanism; 123 indicates the third safeguard mechanism; 124 indicates the fourth safeguard mechanism; 210, 220, 230, and 240 separately indicate the first, the second, the third, and the fourth lateral pieces; 300 indicates the implantable sleeve, 320 indicates the connecting end; 500 indicates the first joining rod, and 600 indicates the second joining rod.

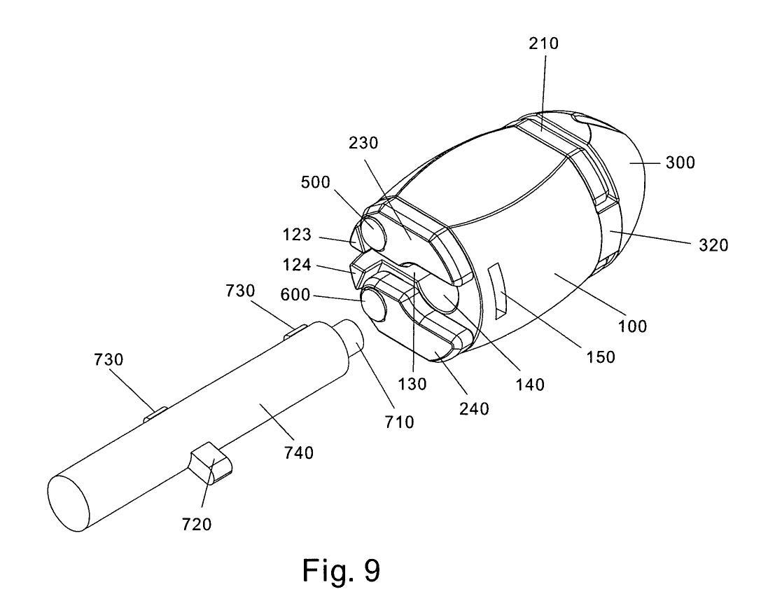

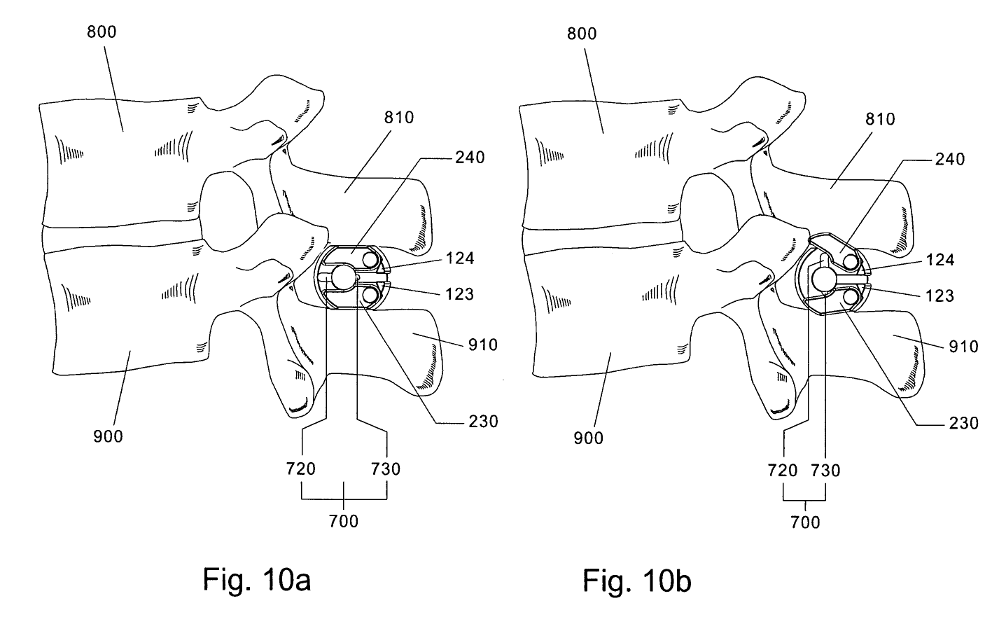

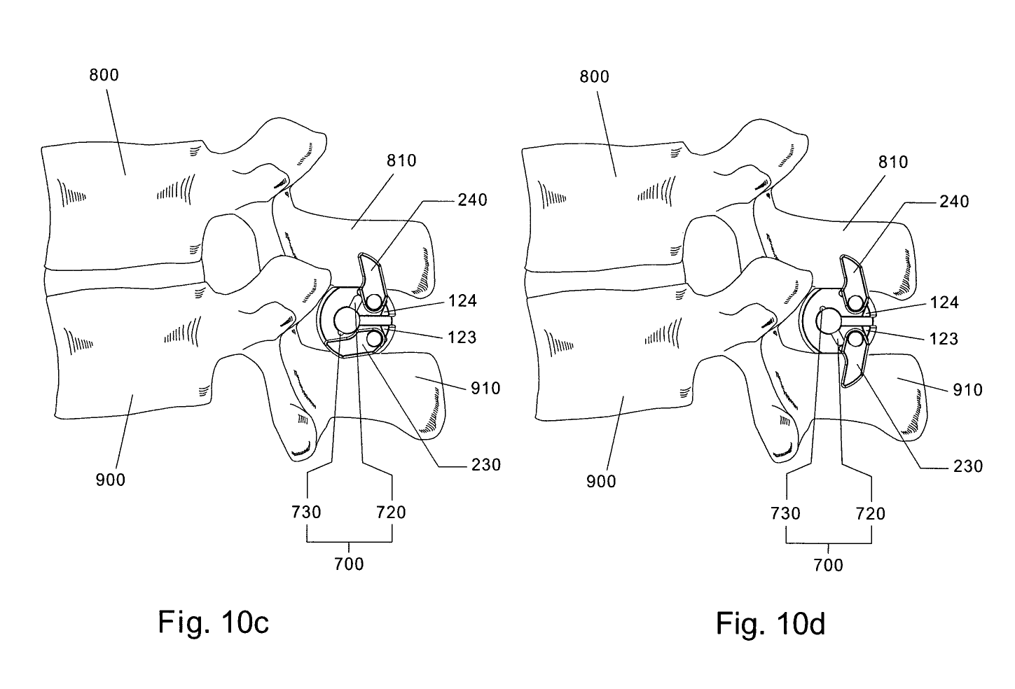

FIG. 2b is a perspective view that shows the lateral pieces of FIG. 1 in an extended state, wherein the numbers 100, 130, 140, 150, 123, 124, 210, 220, 230, 240, 300, 210, 500, and 600 refer to the structures denoted by the same numbers in FIG. 2a. Refer to FIGS. 9 and 10 for the extending mechanisms of the apparatus.

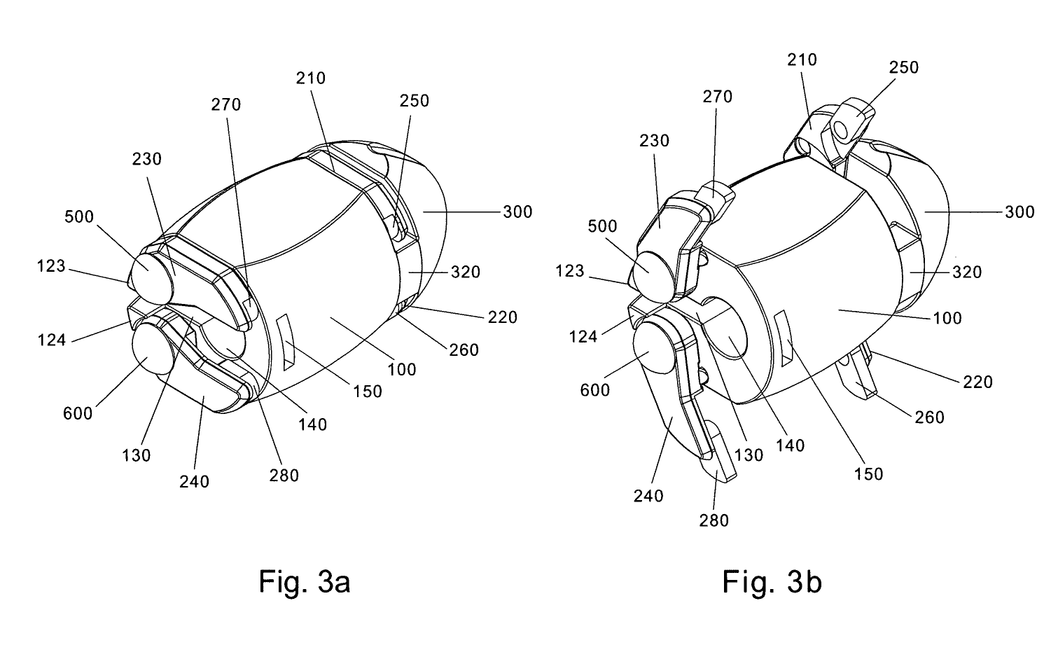

FIG. 3a is a perspective view that shows an interspinous spinal fixation apparatus in a folded state according to another preferred embodiment of the invention, which is similar to the apparatus shown in FIG. 1 to FIG. 2b except that the lateral pieces are further fitted with auxiliary lateral pieces, wherein like elements or parts are designated by like numerals. The number 100 is the main body; 130 is the opening; 140 is the pivot hole; 150 is the fixing mechanism; 123 is the third safeguard mechanism, and 124 is the fourth safeguard mechanism; 210, 220, 230, and 240 are the first, the second, the third, and the fourth lateral pieces; 250, 260, 270, and 280 are the first, the second, the third, and the fourth auxiliary lateral pieces; 300 is the implantable sleeve, 320 is the connecting end; 500 is the first joining rod, and 600 is the second joining rod.

FIG. 3b is a perspective view that shows the interspinous spinal fixation apparatus of FIG. 3a in an extended state, wherein the numbers 100, 130, 140, 150, 123, 124, 210, 220, 230, 240, 250, 260, 270, 280, 300, 320, 500, and 600 refer to the structures denoted by the same numbers in FIG. 3a.

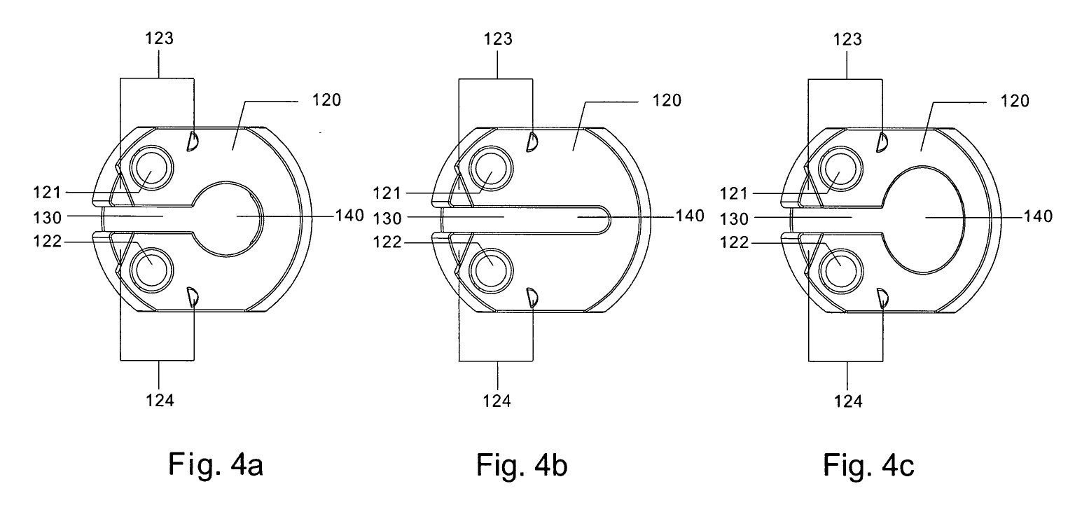

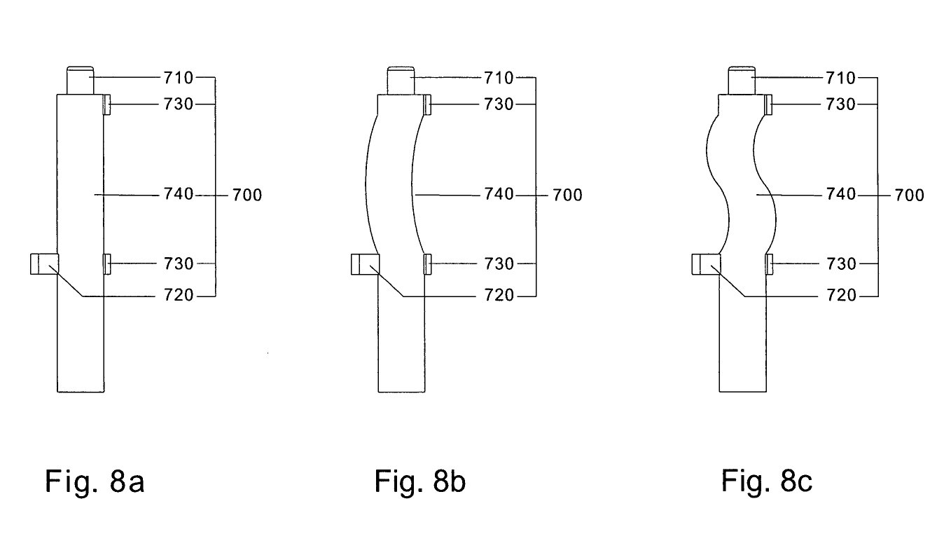

FIGS. 4a, 4b, and 4c are close schematic views that show three different shapes of pivot holes of the interspinous spinal fixation apparatus according to a preferred embodiment of the invention, in which the number 120 is the proximal end; 130 is the opening; 140 is the pivot hole; 123 is the third safeguard mechanism; 124 is the fourth safeguard mechanism; 121 is the third joining point; 122 is the fourth joining point. It can be observed in FIG. 4a that the pivot hole 140 is circular, while the pivot hole 140 of FIG. 4b is of identical width as the opening 130, and pivot hole 140 of FIG. 4c is elliptical. All of the pivot holes 140 shown in FIGS. 4a, 4b, and 4c allow an actuator to be inserted thereinto, and the lateral pieces may then be extended by rotating the actuator (please refer to FIG. 8a to FIG. 10b).

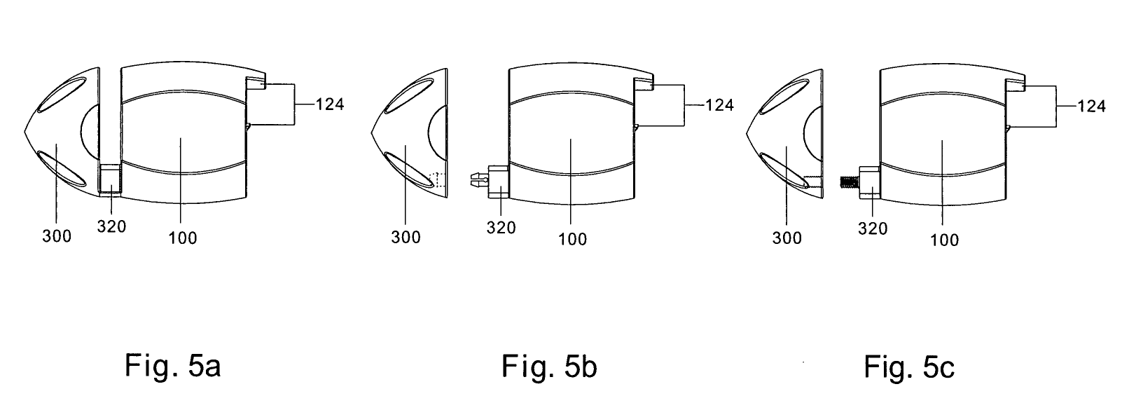

FIGS. 5a, 5b, and 5c are schematic views that show three different ways for combining the main body and the implantable sleeve according to a further preferred embodiment of the invention, wherein the number 100 is the main body; 124 is the fourth safeguard mechanism; 300 is the implantable sleeve, and 320 is the connecting end. Moreover, FIG. 5a shows the implantable sleeve 300 is integrally formed with the main body 100; while FIG. 5b shows the implantable sleeve 300 is combined with the main body 100 by clasping the connecting end 320 into the implantable sleeve 300, and FIG. 5c shows the implantable sleeve 300 is combined with the main body 100 by screwing the connecting end 320 into the implantable sleeve 300.

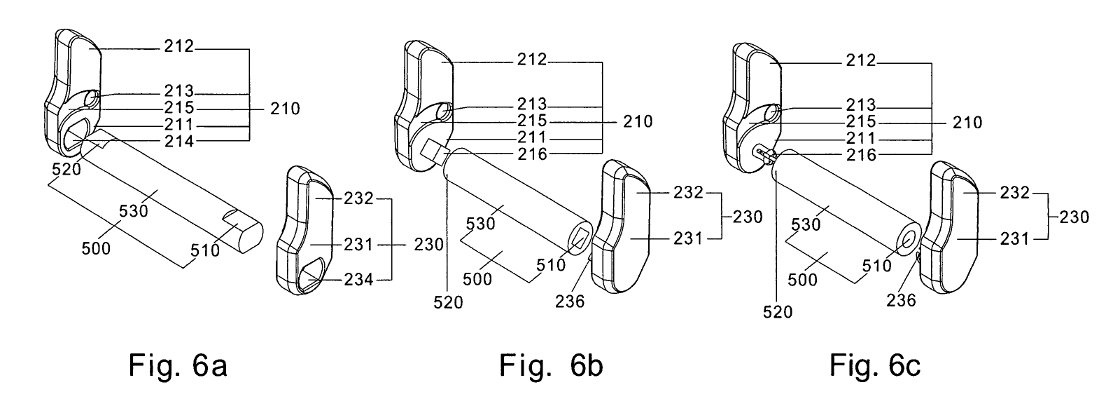

FIGS. 6a, 6b, and 6c are schematic views that show three different ways for combining the lateral pieces and the joining rod according to a further preferred embodiment of the invention, wherein 210 is the first lateral piece; 211, 212, 213, 214, 215, and 216 are respectively the joining end, the extending end, the securing hole, the joining hole, the groove, and a protruding block of the first lateral piece 210. In addition, 230 is the third lateral piece; 231, 232, 234, and 236 are respectively the joining end, the extending end, the joining hole, and the protruding block of the third lateral piece 230; 500 is the first joining rod; 510 and 520 are respectively the upper joining end and the lower joining end of the first joining rod 500, while 530 is the rod body thereof. FIG. 6a indicates that for the purpose of combination, the joining hole 214 of the first lateral piece 210 and the joining hole 234 of the third lateral piece 230 has parallel upper and lower surfaces, and left and right surfaces that are arc-shaped, and the rod body 530 of the joining rod 500 has two opposite sides formed as planar surfaces. FIG. 6b indicates that for the purpose of combination, the protruding block 216 of the first lateral piece 210 and the protruding block 236 of the third lateral piece 230 are shaped as columns, so as to be fitted into the upper joining end 510 and the lower joining end 520 of the first joining rod 500. FIG. 6c indicates that for the purpose of combination, the protruding block 216 of the first lateral piece 210 and the protruding block 236 of the third lateral piece 230 are shaped as pins, so as to be fitted into the upper joining end 510 and the lower joining end 520 of the first joining rod 500.

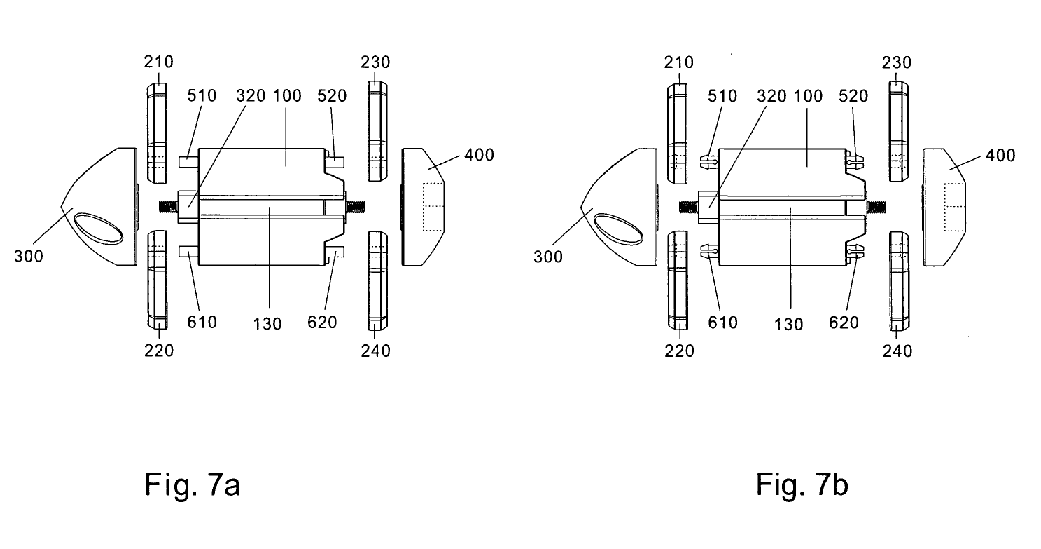

FIGS. 7a and 7b are schematic views that show two different ways for combining the lateral pieces and the main body according to a further preferred embodiment of the invention, wherein 100 is the main body; 130 is the opening; 210, 220, 230, and 240 are the first, the second, the third, and the fourth lateral pieces; 300 is the implantable sleeve; 320 is the connecting end, and 400 is a fixing sleeve. In addition, FIG. 7a shows the first lateral piece 210 and the third lateral piece 230 are respectively connected to the upper joining end 510 and the lower joining end 520 of the first joining rod 500; the second lateral piece 220 and the fourth lateral piece 240 are respectively connected to the upper joining end 610 and the lower joining end 620 of the second joining rod 600. The implantable sleeve 300 and the fixing sleeve 400 are both combined with the main body 100 by screwing, so as to further secure the first lateral piece 210, the second lateral piece 220, the third lateral piece 230, and the fourth lateral piece 240 on the main body 100. The first and third lateral pieces 210 and 230 are allowed to rotate about the first joining rod 500; and the second and the fourth lateral pieces 220 and 240 are allowed to rotated about the second joining rod 600. FIG. 7b shows the first lateral piece 210 and the third lateral piece 230 are respectively connected to the upper joining end 510 and the lower joining end 520 of the first joining rod 500 by clasping; the second lateral piece 220 and the fourth lateral piece 240 are respectively connected to the upper joining end 610 and the lower joining end 620 of the second joining rod 600 by clasping. The implantable sleeve 300 and the fixing sleeve 400 are both combined with the main body 100 by screwing, so as to further secure the first lateral piece 210, the second lateral piece 220, the third lateral piece 230, and the fourth lateral piece 240 on the main body 100. The first and third lateral pieces 210 and 230 are allowed to rotate about the first joining rod 500; and the second and the fourth lateral pieces 220 and 240 are allowed to rotated about the second joining rod 600.

FIGS. 8a, 8b, and 8c are schematic views that illustrate three different actuators according to a further preferred embodiment of the invention, wherein 700 is an actuator; 710 is a fixing block; 720 is a rotating block; 730 is an aligning block, and 740 is a rod body. Moreover, the fixing block 710 is inserted into the implantable sleeve so as to support the actuator 700 in the main body, and the aligning block 730 is inserted into the opening of the main body so as to align the actuator in the main body; please refer to FIGS. 9 and 10 for methods of operating the actuator 700. FIG. 8a shows the rod body is shaped as a column, and FIG. 8b shows the rod body is arc-shaped, so as to increase friction between the rod body and the main body while inserting the rod body; FIG. 8c shows the rod body is S-shaped, so as to increase friction between the rod body and the main body while inserting the rod body.

FIG. 9 is a schematic view that illustrates a way for implementing the interspinous spinal fixation apparatus and the actuator according to a further preferred embodiment of the invention, wherein 100 is the main body; 120 is the proximal end of the main body 100; 123 and 124 are respectively the third and the fourth safeguard mechanisms of the proximal end 120; 130 is the opening of the main body 100; 140 is the pivot hole of the main body 100; 150 is the fixing mechanism of the main body 100; 210, 220, 230, and 240 are the first, the second, the third, and the fourth lateral pieces; 300 is the implantable sleeve; 320 is the connecting end of the implantable sleeve 300, in which the implantable sleeve 300 is integrally formed with the main body 100 via the connecting end 320; 500 and 600 are the first and the second joining rods, respectively; 700 is the actuator; 710 is the fixing block; 720 is the rotating block; 730 is the aligning block, and 740 is the rod body.

FIGS. 10a, 10b, 10c, and 10d are schematic views that illustrate the interspinous spinal fixation apparatus being implemented between neighboring vertebrae according to a further preferred embodiment of the invention, wherein 120 is the proximal end of the main body; 123 and 124 are respectively the third and the fourth safeguard mechanisms of the proximal end 120; 130 is the opening of the main body; 230 and 240 are respectively the third and the fourth lateral pieces; 700 is the actuator; 710 is the fixing block; 720 is the rotating block; 800 is an upper vertebra; 810 is a spinous process; 900 is a lower vertebra, and 910 is also a spinous process. FIG. 10a indicates that the interspinous spinal fixation apparatus of the invention is inserted between the spinous process 810 of the upper vertebra 800 and the spinous process 910 of the lower vertebra 900. FIG. 10b shows that the actuator 700 may be rotated, and the fourth lateral piece 240 is subsequently rotated by using the rotating block 720. FIG. 10c shows that the actuator 700 may be rotated, and the fourth lateral piece 240 is subsequently rotated to the extended position by using the rotating block 720. FIG. 10d shows that the actuator 700 may be rotated, and the third lateral piece 230 is subsequently rotated to the extended position by using the rotating block 720.

The present invention has been described with the preferred embodiments thereof and it is understood that many changes and modifications to the described embodiments can be carried out without departing from the scope and the spirit of the invention that is intended to be limited only by the appended claims.

What is claimed is:

1. An interspinous spinal fixation apparatus, comprising:
a main body having an end thereof as a distal end, and another end thereof as a proximal end, wherein a cross section of the main body is of an annular or quasi-annular structure, and a first joining point and a second joining point are provided at the distal end that are symmetric to each other relative to an opening of the annular or quasi-annular structure, while a third joining point and a fourth joining point are provided at the proximal end that that are symmetric to each other relative to the opening of the annular or quasi-annular structure; and
at least four lateral pieces, including a first lateral piece, a second lateral piece, a third lateral piece, and a fourth lateral piece, and an end of each of the lateral pieces is an extending end, whereas another end thereof is an joining end, wherein the joining ends of the first, the second, the third, and the fourth lateral pieces are used for joining with the first, the second, the third, and the fourth joining points of the main body, such that the first and the second lateral pieces can rotate axially about the first and the second joining points, thereby rotating from a folded position on the main body to an extended position.

2. The interspinous spinal fixation apparatus of claim 1, wherein the annular or quasi-annular structure is a C-shaped ring or a quasi C-shaped ring structure.

3. The interspinous spinal fixation apparatus of claim 2, wherein the annular or quasi-annular structure is a C-shaped ring structure.

4. The interspinous spinal fixation apparatus of claim 3, wherein a pivot hole of the C-shaped ring structure is elliptical or circular.

5. The interspinous spinal fixation apparatus of claim 1, wherein the distal end of the main body has a first safeguard mechanism and a second safeguard mechanism, and the proximal end of the main body has a third safeguard mechanism and a fourth safeguard mechanism, so as to secure the first, the second, the third, and the fourth lateral pieces at the extended position.

6. The interspinous spinal fixation apparatus of claim 1, wherein the main body may further include a fixing mechanism for securing the main body while the lateral pieces are being rotated, such that the main body is prevented from loosening off an interspinous space.

7. The interspinous spinal fixation apparatus of claim 1, wherein the distal end of the main body may further include an implantable sleeve.

8. The interspinous spinal fixation apparatus of claim 7, wherein the implantable sleeve is integrally formed with the main body.

9. The interspinous spinal fixation apparatus of claim 1, wherein the proximal end of the main body may further include a fixing sleeve.

10. The interspinous spinal fixation apparatus of claim 1, wherein the joining ends of the first, the second, the third, and the fourth lateral pieces are joined with the first, the second, the third, and the fourth joining points of the main body by clasping, screwing, or locking, such that the first, the second, the third, and the fourth lateral pieces can be rotated axially about the first, the second, the third, and the fourth joining points on the main body.

11. The interspinous spinal fixation apparatus of claim 1, wherein the first and the third joining points include a cylindrical hole that passes through the first and the third joining points and penetrates the main body, and a joining rod fitted through the cylindrical hole, wherein the joining ends of the first and the third lateral pieces are connected to two ends of the joining rod by clasping, screwing, and locking, thereby allowing the extending ends of the first and the third lateral pieces to be rotated axially about the joining rod.

12. The interspinous spinal fixation apparatus of claim 1, wherein the second and the fourth joining points include a cylindrical hole that passes through the second and the fourth joining points and penetrates the main body, and a joining rod fitted through the cylindrical hole, wherein the joining ends of the second and the fourth lateral pieces are connected to two ends of the joining rod by clasping, screwing, and locking, thereby allowing the extending ends of the second and the fourth lateral pieces to be rotated axially about the joining rod.

13. The interspinous spinal fixation apparatus of claim 1, wherein the first, the second, the third, and the fourth lateral pieces may further include a first auxiliary lateral piece, a second auxiliary lateral piece, a third auxiliary lateral piece, and a fourth auxiliary lateral piece, respectively.
